(19) United States
(12) Patent Application Publication
(10) Pub. No.: US 2021/0060550 A1
(43) Pub. Date: Mar. 4, 2021

(54) DEVICES AND METHODS FOR DETECTION OF MICROORGANISMS

(71) Applicant: MICROBIOSENSOR LIMITED, Manchester (GB)

(72) Inventors: Curtis Bryce DOBSON, Manchester (GB); Nishal GOVINDJI-BHATT, Manchester (GB); Duncan HENDERSON, Manchester (GB)

(21) Appl. No.: 16/649,036

(22) PCT Filed: Sep. 25, 2018
(86) PCT No.: PCT/GB2018/052727

§ 371 Date: Mar. 19, 2020

(30) Foreign Application Priority Data
Sep. 28, 2017 (GB) 1715724.9

(51) Int. Cl. B01L 3/00; C12Q 1/04; C12M 1/34

(52) U.S. Cl. CPC B01L 3/50853; C12Q 1/045; C12M 1/34; B01L 2300/0864; B01L 2300/047; B01L 2300/0832; B01L 2300/045

(57) ABSTRACT

A device for determining the presence of microorganisms in a sample and the antimicrobial sensitivity thereof, the device comprising multiple chambers including: —a first control chamber; and —a first test chamber; wherein said first control chamber comprises: —media and/or nutrients that support or encourage microbial growth; and —a tetrazolium indicator compound; and said first test chamber comprises: —media and/or nutrients that support or encourage microbial growth; —a tetrazolium indicator compound; and —a first selective agent which inhibits the growth and/or replication of one or more specific microorganisms.

The present invention relates to devices for detecting the presence of pathogens in biological samples and to methods and uses relating thereto. In particular the invention relates to a device for detecting pathogens indicative of a urinary tract infection in a urine sample, and the antimicrobial sensitivity thereof.

Urinary tract infections (UTIs) can be very painful, cause significant distress and may have serious consequences if left untreated. Unfortunately many vulnerable patients are unable to recognise the symptoms of an infection or articulate their discomfort. UTIs are common in care homes for the elderly or infirm. People who have urinary tract infections may become confused, angry or delirious. However such symptoms may be difficult to distinguish from those of other conditions that may also be present, for example dementia.

Even when a UTI is suspected it can be difficult and slow using current methods to identify the specific pathogen causing the infection. This can cause delays in a patient receiving the correct treatment, leading to prolonged suffering and an increased risk of complications.

The present invention seeks to provide a device which facilitates the rapid and accurate detection of bacteria in a sample and their antibiotic sensitivity.

According to a first aspect of the present invention there is provided a device for determining the presence of microorganisms in a sample and the antimicrobial sensitivity thereof, the device comprising multiple chambers including:
a first control chamber; and
a first test chamber;

wherein said first control chamber comprises:
media and/or nutrients that support or encourage microbial growth; and
a tetrazolium indicator compound;

and said first test chamber comprises:
media and/or nutrients that support or encourage microbial growth;
a tetrazolium indicator compound; and
a first selective agent which inhibits the growth and/or replication of one or more specific microorganisms.

According to a second aspect of the present invention there is provided a method of determining the presence of microorganisms in a sample and the antimicrobial sensitivity thereof, the method comprising:
(a) contacting the sample with a first control composition comprising:
   media and/or nutrients that support or encourage microbial growth; and
   a tetrazolium indicator compound; and
(b) contacting the sample with a first test composition comprising:
   media and/or nutrients that support or encourage microbial growth;
   a tetrazolium indicator compound; and
   a first selective agent which inhibits the growth and/or replication of one or more specific microorganisms; and
(c) observing said first control composition and said first test composition.

In the method of the second aspect step (a) may be carried out before step (b) or step (b) may be carried out before step (a). Preferably steps (a) and (b) are carried out at the same time and portions of the same sample are separately contacted with the separate compositions.

The first control composition and the first test composition are suitably not mixed together.

Step (c) is carried out after steps (a) and (b).

Preferably the method of the second aspect involves delivering the sample into a device of the first aspect and observing the device. Suitably the first control composition is provided in the first control chamber and the first test composition is provided in the first test chamber.

Preferred features of the first and second aspects of the invention will now be described.

The present invention provides a device and method for the detection and/or identification of microorganisms in a sample. Suitably the invention is used for the detection and/or identification of pathogenic microorganisms. The presence of such pathogenic microorganisms in the sample is indicative of infection with said pathogens.

The sample is suitably a sample of bodily fluid. Suitably the sample is selected from blood or components thereof, mucus, saliva, urine, pus, sputum, wound exudate, pleural fluid and peritoneal dialysis effluent.

Preferably the sample is a liquid sample. Most preferably the sample is a urine sample. Preferably the present invention relates to a device and/or method for the detection and/or identification of pathogens indicative of a urinary tract infection (UTI) in a urine sample.

In some embodiments the device of the first aspect includes means for delivering a portion of the sample into each chamber.

Such means suitably includes a common inlet which is shaped to direct the sample into each of the chambers. This enables an unskilled user to pour a sample into the device without the need for specialist training.

The device may also be provided with sealing means. The sealing means suitably prevents the sample from leaking from the device. Preferably the sealing means also prevents transfer of material between chambers.

The sealing means may be used to effectively “lock” the device in a closed position. This prevents leakage and contamination.

In some embodiments the sample may be diluted prior to delivery into the first control composition and the first test composition. In such embodiments the method of the second aspect suitably involves a step prior to step (a) and step (b) of diluting the sample, and step (a) involves contacting the diluted sample with a first control composition and step (b) involves contacting the diluted sample with a first test composition.

The sample may be diluted with any suitable composition. Preferably the sample is diluted by admixture with an aqueous composition. Preferably the sample is diluted by admixture with an aqueous buffer. Suitably buffers will be known to the person skilled in the art and include, for example phosphate and borate buffered saline.

The dilution ratio will depend on the nature of the sample. The selection of an appropriate dilution ratio will be within the competence of the skilled person. In the case of a urine sample typical dilution ratios are 1:5 to 1:500 and preferred dilution ratios are 1:10 to 1:100. [okay?]

In preferred embodiments in which the method of the second aspect is carried out using a device of the first aspect, the step of diluting the sample may be carried out before or after the sample is delivered into the device.

For example the sample may be first mixed with a diluent, for example an aqueous buffer, before it is delivered to the device. In some embodiments a separate device for diluting the sample may be provided. For example a syringe may be provided which is preloaded with a diluent (such as an aqueous buffer) and can further accommodate a predetermined amount of sample.

In some preferred embodiments dilution of the sample may be carried out within the device.

In some embodiments the device of the first aspect further comprises a dilution reservoir.

The dilution reservoir is suitably configured to hold a diluent (such as an aqueous buffer) and to receive a portion of the sample.

Preferably the dilution reservoir is configured to be able to deliver equal volumes of diluted sample to each chamber of the device.

Suitably there is a closable aperture connecting each chamber to the reservoir. Suitably the device further comprises a switch which allows opening and closing of all apertures on each chamber concurrently.

Suitably in the method of the second aspect a device may be provided comprising a dilution reservoir in which the apertures connecting each chamber to the device are initially closed and a diluent (such as an aqueous buffer) is provided in the dilution reservoir. Preferably a suitable amount of sample is delivered into the reservoir to provided the desired dilution ratio. Suitable the switch is used to open all of the apertures thereby delivering the contents of the reservoir to each chamber.

The first control chamber, the first control composition, the first test chamber and the first test composition comprise media and/or nutrients that support or encourage microbial growth.

In the device of the first aspect the same or different media and/or nutrients may be used in each chamber.

Preferably the same media and/or nutrients are used in each chamber.

In the method of the second aspect the same or different media and/or nutrients may be used in each composition.

Preferably the same media and/or nutrients are used in each composition.

Preferably the media and/or nutrients are selected to:
(i) maintain viable micro-organisms in the reporting means and in some embodiments supports or encourages microbial growth and/or division;
(ii) take into account whether a narrow spectrum or broad spectrum of microorganisms needs to be maintained and detected;
(iii) not result in false triggering of the indicator in the absence of microorganisms;
(iv) not degrade or inactivate any of other components.

Examples of media/nutrients which may be used according to the invention include Mueller Hinton, brain heart infusion broth (BHI) and Wilkins Chalgren media. Preferably the media/nutrients is selected from brain heart infusion and Wilkins Chalgren media. It is most preferred that Wilkins Chalgren media is used in each chamber or each composition.

The first control chamber, the first control composition, the first test chamber and the first test composition comprise a tetrazolium indicator compound.

In the device of the first aspect the same or different tetrazolium indicator compound may be used in each chamber.

Preferably the same tetrazolium indicator compound is used in each chamber.

In the method of the second aspect the same or different tetrazolium indicator compound may be used in each composition.

Preferably the same tetrazolium indicator compound is used in each composition.

The tetrazolium indicator compound may be any compound that undergoes an observable change when microorganisms are present.

The observable change may be a change in light absorption, precipitate formation, bubble formation, a temperature change or other measurable quality.

Preferably the observable change is a colour change. Suitably the indicator compound is a different colour in the presence of microorganisms than it is when no microorganisms are present. The indicator compound suitably has an initial colour before the reporting means composition is contacted with the sample. If microorganisms are present in the sample the indicator compound preferably undergoes a colour change. The indicator compound may change from colourless to coloured, from coloured to colourless, or from a first colour to a second colour which is different to the first colour.

The skilled person will appreciate that the colour change is suitably due to a change in the structure of the indicator compound which affects the chromophore region.

Preferably the indicator changes from colourless to coloured in the presence of microorganisms.

The indicator compound is preferably a redox indicator.

The indicator compound is a tetrazolium compound.

Suitable tetrazolium compounds include MTT (3-(4,5-Dimethylthiazol-2-yl)-2,5-diphenyltetrazolium bromide); XTT (2,3-bis-(2-methoxy-4-nitro-5-sulfophenyl)-2H-tetrazolium-5-carboxanilide); MTS (3-(4,5-dimethylthiazol-2-yl)-5-(3-carboxymethoxyphenyl)-2-(4-sulfophenyl)-2H-tetrazolium); water soluble tetrazolium salts (WST) such as WST-1, WST-3, WST-4, WST-5, WST-7, WST-8, WST-9, WST-10 or WST-11; indonitrotetrazolium chloride (INT); Nitrobluetetrazolium (NBT); Tetranitro blue tetrazolium (TNBT); Thiocarbamyl nitro blue tetrazolium (TCNBT); Tetrazolium red (TR); tetrazolium violet (TV); neotetrazolium chloride; and 5-cyano-2,3-ditolyl tetrazolium chloride (CTC).

In some embodiments the indicator compound is a water soluble tetrazolium salt (WST).

One preferred WST that the indicator compound is WST-9 or a derivative thereof. WST-9 has the chemical formula: 2-(4-Nitrophenyl)-5-phenyl-3-[4-(4-sulfophenylazo)-2-sulfophenyl]-2H-tetrazolium, monosodium salt and the chemical structure:

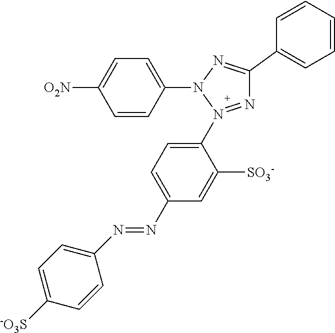

Most preferably the tetrazolium indicator compounds is tetrazolium violet.

Tetrazolium violet has the structure:

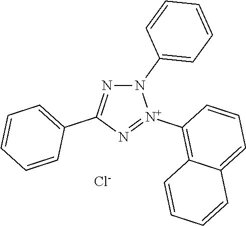 [SMILES: c1ccc(-c2nn(-c3ccccc3)[n+](-c3cccc4ccccc34)n2)cc1 ; [Cl-]]

The media and/or nutrients used in the reporting means is preferably a media or broth that does not cause tetrazolium violet (TV) to change colour when the media or broth is incubated with tetrazolium violet at 37° C. overnight. Alternatively, the media and/or nutrients used in the reporting means is preferably a media or broth that does not cause tetrazolium violet to change colour when the media or broth is incubated with tetrazolium violet at 24° C. for 8 hours or up to 24 hours. The colour change of tetrazolium violet may be assayed by measuring, over time, the Optical Density of media solutions mixed with tetrazolium violet.

The first test chamber or the first test composition further comprises a selective agent which inhibits the growth and/or replication of one or more specific microorganisms.

By “selective agent” we mean an agent that will arrest replication, decrease growth, metabolism and/or increase death of certain microorganisms and will not affect the growth or death rate of others. It will be appreciated that a sufficient amount of the selective agent should be included that will prevent any activation of the device by microorganisms that are sensitive to it.

For the avoidance of doubt when we refer to activation of the device we mean that the tetrazolium indicator compound has undergone an observable change, suitably a colour change. The skilled person will appreciate that the observable change will occur once a threshold concentration of microorganisms is reached.

In this specification when we make reference to a selective agent being “effective” against a particular microorganism(s) we mean that the selective agent inhibits the growth, metabolism and/or replication of said microorganism(s).

By inhibiting growth and/or replication we mean to include agents that kill the microorganism, reduce their growth and/or metabolism, retard their growth and/or restrict or destroy their ability to reproduce.

The selective agents used in the present invention are suitable for allowing discrimination between different types of microorganism when the activation of the control chamber and the test chamber or the control composition and the test composition are compared.

In one embodiment a selective agent is chosen that has broad spectrum activity against bacteria, but which is selective for bacteria over other types of microorganism.

In another embodiment the selective agent may be an agent with narrow spectrum activity (for instance an agent that only has antibiotic activity against a limited number of species of bacteria). Such narrow spectrum selective agents are useful as selective agents when a device is designed where the user expects a sample to contain a specific microorganism. By way of examples sodium nalidixate is a narrow spectrum agent which is used against Pseudomonas spp. It may be used as a selective agent in the test channel/composition in devices/method designed to identify whether or not there is a Pseudomonas spp infection.

In a further embodiment the selective agent prevents the growth of Gram negative microorganisms. According to this embodiment, activation of the test chamber/composition will indicate that a subject is infected with a Gram positive microorganism because the selective agent failed to prevent activation of the indicator compound. A user will establish that there is a Gram negative infection if the test chamber/composition is not activated. Polymyxin B sulphate, gentamycin or monobactam compounds are antibiotics used primarily for Gram-negative infections and which may be used according to this embodiment of the invention.

In some embodiments, the selective agent prevents the growth of Gram positive microorganisms. According to this embodiment, activation of the test chamber/composition will indicate that a subject is infected with a Gram negative microorganism because the selective agent failed to prevent an observable change in the indicator compound. However, a user will establish that there is a Gram positive infection if the test chamber/composition is not activated.

Fusidin (Fusidic acid) may be used as a selective agent for establishing whether or not there is a Gram positive infection. It is a bacteriostatic antibiotic which is effective primarily against Gram-positive bacteria.

A selective agent which inhibitors the growth of Gram positive organisms is vancomycin.

The present invention is preferably used for the detection and/or identification of pathogens indicative of a UTI.

In many cases UTIs are caused by a bacterial infection, especially with Gram negative bacteria.

Thus in preferred embodiments the selective agent selectively inhibits the growth and/or replication of one or more species of Gram negative bacteria.

In the present invention if the first control channel is activated this shows that there are microorganisms present in the sample—if the test sample is activated this shows that the selective agent is not effective against the microorganisms present in the sample (or is not effective against all of the microorganisms present therein). By effective against we mean that the agent inhibits the growth and/or replication of the microorganisms present in the sample.

If the test channel is not activated this indicates that the selective agent is effective against the microorganisms present in the sample. This would therefore suggest that the selective agent could be used in a treatment against a pathogenic infection with said microorganisms.

Preferably the first selective agent is an antibiotic compound that is effective against one or more species of Gram negative bacteria.

Suitably the first selective agent is selected from fosfomycin, cephalosporins including cephalexin, cefuroxime, cefoxitin and cefotaxime; sulfamethoxazole-trimethoprim, quinolones including gentamicin; penicillins including amoxycillin, amikacin and aminopenicillins including ampicillin; beta lactam inhibitors including coamoxyclav and tazobactam; temocillin, tetracylines including doxycycline; carbapenems including meropenem and ertapenem; fluoroquinolones including ciprofloxacin; nitrofurantoin, pivmecillnam, piperacillin and trimethoprim.

Preferably the first selective agent is selected from ciprofloxacin, nitrofurantoin, pivmecillnam, trimethoprim and cephalexin.

In some embodiments the first test chamber or the first test composition may comprise a mixture of two or more selective agents. As such the device may be used to test the sensitivity of any microbes in the sample to the combination of selective agents.

In some embodiments the device of the first aspect further comprises one or more further test chambers wherein the or each further test chamber comprises media and/or nutrients that support or encourage microbial growth; a tetrazolium indicator compound; and a selective agent which inhibits the growth and/or replication of one or more specific microorganisms.

In some embodiments the or each further test chamber may comprise a mixture of two or more selective agents.

In some embodiments step (b) of the method of the second aspect further comprises contacting the sample with one or more further test compositions wherein the or each further test composition comprises: media and/or nutrients that support or encourage microbial growth; a tetrazolium indicator compound; and a selective agent which inhibits the growth and/or replication of one or more specific microorganisms.

In some embodiments the or each further test composition may comprise a mixture of two or more selective agents.

Suitably the device of the first aspect comprises between 1 and 20 further test chambers, preferably between 1 and 10, more preferably between 1 and 8, suitably between 2 and 6, preferably between 3 and 7, for example 4, 5 or 6.

Suitably step (b) of the method of the second aspect involves contacting the sample with between 1 and 20 further test compositions, preferably between 1 and 10, more preferably between 1 and 8, suitably between 2 and 6, preferably between 3 and 7, for example 4, 5 or 6.

Suitably the device of the first aspect includes from 1 to 10, preferably 2 to 8, more preferably 4 to 6 test chambers.

Suitably step (b) of the method of the second aspect involves contacting the sample with 1 to 10, preferably 2 to 8, more preferably 4 to 6 test compositions.

The media and/or nutrients that support or encourage microbial growth used in the or each further test chamber of the device of the first aspect may be the same or different to that used in the first test chamber.

When more than two further test chambers are included in the device each may have the same or different media and/or nutrients.

Preferably each test chamber (i.e. the first test chamber and the one or more further test chambers) comprises the same media and/or nutrients that support and/or encourage microbial growth.

Preferably the media and/or nutrients in each test chamber is selected from Mueller Hinton, brain heart infusion broth (BHI) and Wilkins Chalgren media. Preferably the media/nutrients is selected from brain heart infusion and Wilkins Chalgren media. It is most preferred that Wilkins Chalgren media is used in each chamber.

The media and/or nutrients that support or encourage microbial growth used in the or each further test composition of the method of the second aspect may be the same or different to that used in the first test composition.

When two or more further test compositions are used in the method of second aspect each may have the same or different media and/or nutrients.

Preferably each test composition (i.e. the first test composition and the one or more further test compositions) comprises the same media and/or nutrients that support and/or encourage microbial growth. Suitably the media and/or nutrients used in each test composition is selected from Mueller Hinton, brain heart infusion broth (BHI) and Wilkins Chalgren media. Preferably the media/nutrients is selected from brain heart infusion and Wilkins Chalgren media.

It is most preferred that Wilkins Chalgren media is used in each composition.

The tetrazolium indicator compound used in the or each further test chamber of the device of the first aspect may be the same or different to that used in the first test chamber.

When more than two further test chambers are included in the device each may comprise the same or different tetrazolium indicator compound.

Preferably each test chamber (i.e. the first test chamber and the one or more further test chambers) comprises the same tetrazolium indicator compound.

Suitable compounds are as previously described herein.

Preferably each test chamber of the device comprises tetrazolium violet.

The tetrazolium indicator compound used in the or each further test composition of the method of the second aspect may be the same or different to that used in the first test composition.

When step (b) of the method of the second aspect involves contacting the sample into two or more further test compositions each may comprise the same or different tetrazolium indicator compound.

Preferably each test composition (i.e. the first test composition and the one or more further test compositions) comprises the same tetrazolium indicator compound.

Suitable compounds are as previously described herein.

Preferably each test composition comprises tetrazolium violet.

The selective agent which inhibits the growth and/or replication of one or more specific microorganisms used in the or each further test chamber of the device of the first aspect may be the same or different to that used in the first test chamber.

When two or more further test chambers are included in the device each may include the same or different selective agent which inhibits the growth and/or replication of one or more specific microorganisms.

Preferably each test chamber comprises a different selective agent which inhibits the growth and/or replication of one or more specific microorganisms.

When two or more further test compositions are used in the method of the second aspect each may include the same or different selective agent which inhibits the growth and/or replication of one or more specific microorganisms.

Preferably each test composition comprises a different selective agent which inhibits the growth and/or replication of one or more specific microorganisms.

The device and method of the present invention suitably involve the use of multiple chambers/compositions. Each test chamber or test composition has a different capacity to inhibit the growth and/or replication of one or more specific microorganisms.

The control chamber and control composition do not contain any agent which inhibits microorganisms. Activation of the control chamber/composition is indicative that microorganisms are present but gives no information about the nature of the microorganisms.

Each test chamber includes a selective agent that is able to inhibit specific microorganisms. Activation (for example by a colour change) of the test chamber indicates that the selective agent is not effective against some or all of the microorganisms present in the sample. If a test chamber is not activated this indicates that the selective agent would be effective against the microorganisms present in the sample. Thus when the sample is a clinic sample infected with a microbial pathogen the present invention may be used to determine which compounds may be an effective treatment against the infection.

The provision of multiple chambers/compositions enables the effectiveness of multiple selective agents against microorganisms in the sample to be tested concurrently.

In some embodiments the device of the first aspect comprises at least two test chambers wherein each test chamber includes a different selective agent or combination of selective agents. Preferably each chamber comprises one or more selective agents selected from ciprofloxacin, nitrofurantoin, pivmecillinam, trimethoprim, cephalexin and mixtures thereof.

In some embodiments the device of the first aspect comprises at least three test chambers wherein each test chamber includes a different selective agent or combination of selective agents. Preferably each chamber comprises one or more selective agents selected from ciprofloxacin, nitrofurantoin, pivmecillinam, trimethoprim, cephalexin and mixtures thereof.

In some embodiments the device of the first aspect comprises at least four test chambers wherein each test chamber includes a different selective agent or combination of selective agents. Preferably each chamber comprises one or more selective agents selected from ciprofloxacin, nitrofurantoin, pivmecillinam, trimethoprim, cephalexin and mixtures thereof.

In some embodiments the device of the first aspect comprises at least five test chambers wherein each test chamber includes a different selective agent or combination of selective agents. Preferably each chamber comprises one or more selective agents selected from ciprofloxacin, nitrofurantoin, pivmecillinam, trimethoprim, cephalexin and mixtures thereof.

In one embodiment the device of the present invention comprises five test chambers wherein a first test chamber comprises ciprofloxacin, a second test chamber comprises nitrofurantoin, a third test chamber comprises pivmecillinam, a fourth test chamber comprises trimethoprim, and a fifth test chamber comprises cephalexin.

In some embodiments the device of the first aspect may comprise more than five test chambers.

In some embodiments step (b) of the method of the second aspect involves contacting the sample with at least two test compositions wherein each test composition comprises a different selective agent or combination of selective agents. Preferably each test composition comprises one or more selective agents selected from ciprofloxacin, nitrofurantoin, pivmecillinam, trimethoprim, cephalexin and mixtures thereof.

In some embodiments step (b) of the method of the second aspect involves contacting the sample with at least three test compositions wherein each test composition comprises a different selective agent or combination of selective agents. Preferably each test composition comprises one or more selective agents selected from ciprofloxacin, nitrofurantoin, pivmecillinam, trimethoprim, cephalexin and mixtures thereof.

In some embodiments step (b) of the method of the second aspect involves contacting the sample with at least four test compositions wherein each test composition comprises a different selective agent or combination of selective agents. Preferably each test composition comprises one or more selective agents selected from ciprofloxacin, nitrofurantoin, pivmecillinam, trimethoprim, cephalexin and mixtures thereof.

In some embodiments step (b) of the method of the second aspect involves contacting the sample with at least five test compositions wherein each test composition comprises a different selective agent or combination of selective agents. Preferably each test composition comprises one or more selective agents selected from ciprofloxacin, nitrofurantoin, pivmecillinam, trimethoprim, cephalexin and mixtures thereof.

In one embodiment step (b) of the method of the second aspect involves contacting the sample with five test compositions wherein a first test composition comprises ciprofloxacin, a second test composition comprises nitrofurantoin, a third test composition comprises pivmecillinam, a fourth test composition comprises trimethoprim, and a fifth test composition comprises cephalexin.

In some embodiments the step (b) of the method of the second aspect may involve contacting the sample with more than five test compositions.

The device of the first aspect may include one or more further control chambers. A second control chamber may be provided which is initially empty to enable the user to visualise the sample, preferably a urine sample.

The device may include a further control chamber which has the same contents as the first control chamber. In such embodiments this further control chamber is suitably spatially separated within the device from the first control chamber. The provision of two identical control chambers may facilitate easier comparison with multiple test chambers.

In one especially preferred embodiment the present invention provides a device for detecting and/or identifying microorganisms in a sample, preferably a urine sample, the device comprising: a first control chamber and five test chambers wherein each chamber comprises media and/or nutrients that support or encourage microbial growth and a tetrazolium indicator compound and each test chamber comprises a selective agent; wherein the first test chamber comprises ciprofloxacin as the selective agent, the second test chamber comprises nitrofurantoin as the selective agent, the third test chamber comprises pivmecillinam the selective agent, the fourth test chamber comprises trimethoprim as the selective agent and the fifth test chamber comprises cephalexin as the selective agent.

The device may further comprise an empty chamber and optionally a further control chamber comprising media and/or nutrients that support or encourage microbial growth and a tetrazolium indicator compound.

It will be appreciated that the device according to the invention may be used to detect or identify microorganisms in a variety of different fluids. The device has utility for testing effluents in an industrial or environmental setting. It is preferred that the device is used to test a biological fluid (e.g. bronchial lavage fluid, serum, cerebral spinal fluid, urine and the like) and it is most preferred that the device is used to detect or identify microorganisms in a urine sample.

The chambers of the device may be any suitable vessel that can retain the necessary components and into which a sample fluid may be introduced.

Preferably the device is configured such that the contents of the chambers may be easily observed by a user of the device.

In a preferred embodiment each chamber is configured to receive the sample, preferably a liquid sample. The chambers of the device may be formed from a number of materials that are well known to the art. The chambers may be formed from glass or a plastic material, preferably a transparent material.

In some embodiments the device may consist of multiple conjoined chambers. Suitably the chambers are contained within a suitable casing.

In some preferred embodiments the casing incorporates a colour chart that enables a user to compare the colour of the sample in the chamber with the colour provided on the chart. The colours on the chart will illustrate the colour to be expected in the presence/absence of microorganisms.

Components of each chamber (for example the indicator compounds, media and/or nutrients that support or encourage microbial growth, selective agents etc.) may comprise powders that are inserted directly within the chambers.

However, in preferred embodiments the individual components of each chamber are loaded onto, or into, some kind of vehicle. Such vehicles are useful for designing an optimal method of manufacturing devices according to the invention and can be particularly useful when the device, or at least components thereof, need to be sterilized.

A number of vehicles may be used. For instance, components may be made into concentrated solutions that are applied to filter discs. The filter discs are then dried such that they retain the relevant component and the filter discs then placed within the chambers. According to one embodiment the selective agent may be applied to a filter disc. By way of example, 6 mm Whatman or Oxoid filter discs may be impregnated with of a concentrated stock of the component. These discs should then be dried (e.g. at 37° C. for 18 hours or until completely dry). The dried discs may then be inserted in the relevant chambers.

Alternatively, components of each chamber may be combined with suitable binders and excipients to form tablets and the tablets placed within the channels.

It is preferred that the vehicle is a capsule or capsules. In one embodiment all components of one chamber are retained within one capsule. In another embodiment a chamber may contain more than one capsule with components contained within different capsules.

Capsules used according to the invention should dissolve when contacted with a liquid sample being tested and are also ideally colourless or at least a colour that does not affect the visualisation of the indicator compound. Capsules are well known to the art and a skilled person will be easily able to select a capsule which suits the particular components of the chambers into which they will need to be inserted. Preferred capsules may be formed from hydroxypropyl methylcellulose (HPMC) or gelatine.

The size of capsule used will depend on the amount of each component to be used, and this in turn will depend upon the size of the chamber and the amount of fluid it is designed to retain.

In one embodiment all components are retained within a single capsule. For instance, a capsule may contain tetrazolium violet, Wilkins Chalgren media and optionally an antibiotic selected from ciprofloxacin, nitrofurantoin, pivmecillnam, trimethoprim and cephalexin.

The size of the capsule or other vehicle depends on the sample size and the size of the chamber.

In one embodiment the chamber is designed to receive from 0.5 to 20 mL, preferably 0.8 to 10 mL, for example 1 to 5 mL of sample.

In some embodiments the components of one or more chambers are mixed with excipients and then used to fill capsules. A preferred excipient is polyvinylpyrrolidone (PVP) or a derivative thereof. PVP is preferred as it did not cause false triggering or mask a colour change.

In some preferred embodiments polyvinylpyrrolidone (PVP) is used as an excipient. This may be for example under circumstances where components of the chambers need to be mixed with an excipient (e.g. when tablets or formed or to aid in the filling of capsules).

In some embodiments the or each control chamber comprises polyvinylpyrrolidone.

In some embodiments the or each test chamber comprises polyvinylpyrrolidone.

The components of each chamber of the device of the first aspect or each composition used in step (a) or step (b) of the method of the second aspect may be provided in any suitable form.

They may be provided as a powder, tablet, gel, solution or paste. Preferably the components are provided in solid form.

Preferably each component is provided in powdered form within a capsule. Each capsule may comprise one or more of the individual components.

The components used in each composition used in step (a) and step (b) of the method of the second aspect may be provided in any suitable form. Preferably they are provided in solid form, preferably retained with a capsule or capsules.

The provision of components in solid form, especially in capsules, allows ease of handling and ease of manufacturing. It also means that amounts can be measured more accurately which leads to improved performance. The components also have improved storage stability.

The solid compositions provided in the chambers of the first aspect or used in the method of the second aspect suitably dissolve when admixed with a liquid sample, for example urine. The amount of each component provided in each chamber or composition is suitably determined based on the expected volume of sample with which it is to be contacted to provide a desired concentration of the component within the resultant mixture. This mixture of sample and components may be referred to herein as the “tested fluid”.

The term “tested fluid” is used to refer to the mixture present in each control or test chamber after contact with the sample or formed by contact of each control or test composition with the sample.

The amount of media and/or nutrients that support or encourage microbial growth present in the tested fluid is suitably between 1 and 200 mg/mL, preferably from 10 to 100 mg/mL, more preferably from 20 to 50 mg/mL.

The amount of indicator compound present in the tested fluid is selected to be from 0.01 to 10 mg/mL, suitably from 0.05 to 5 mg/mL, preferably from 0.1 to 1 mg/mL, for example from 0.1 to 0.5 mg/mL.

The amount of selective agent used will depend on the nature of the agent and its potency. Suitably the selective agent is present in an amount of from 0.01 to 500 μg/mL, preferably from 0.05 to 200 μg/mL, more preferably from 0.1 to 100 μg/mL.

The amount of excipient used (for example PVP) will depend on the size of the vehicle, for example a capsule. The selection of an appropriate amount is within the competence of the skilled person.

In the method of the second aspect of the present invention the samples contacted with one or more control compositions in step (a) and one or more test compositions in step (b). Preferably steps (a) and (b) are carried out at the same time, preferably using a device of the first aspect.

Step (c) involves observing the control and test compositions. Preferably step (c) involves observing a device of the first aspect.

Step (c) is carried out after steps (a) and (b). Suitably step (c) is not carried out immediately after steps (a) and (b).

Suitably step (c) is carried out for at least 10 minutes after steps (a) and (b), preferably at least 30 minutes, more preferably at least 60 minutes, for example at least 90 minutes.

Step (c) may be carried out for up to 48 hours after steps (a) and (b), suitably up to 36 hours, preferably up to 24 hours, for example up to 20 hours.

Suitably step (c) is carried out for up to 4 to 18 hours after steps (a) and (b), preferably 6 to 16 hours, for example 8 to 12 hours.

There is suitably a delay between contacting the sample with the compositions (preferably the device) and observing the device. This is to enable any microorganisms in the sample to grow and reproduce. This growth and replication leads to a change in the indicator compound.

In some embodiments the compositions, preferably within a device, are incubated after contact with the sample (i.e. the “tested fluid” in each chamber is incubated).

Incubation is suitably carried out at a temperature of 30 to 39° C., preferably about 37° C., preferably for a period of 4 to 16 hours, preferably 8 to 12 hours. In some embodiments the device may be shaped to fit within an incubator.

In some embodiments the device/compositions may be cooled following incubation, for example to about 4° C. This cooling inhibits microbial activity and thus reduces further activation of the compositions after incubation.

After incubation the tested fluid in each composition/chamber is observed.

By comparing the test chamber/compositions with the control chamber/compositions it is possible to determine whether any microorganisms are present in the sample and if so whether any of the selective agents tested could be used to inhibit the microorganisms.

In step (c) the compositions (preferably within the device) are observed.

As mentioned above the device of the first aspect is suitably configured to enable a user to readily observe the contents of each chamber. For example a readily removable lid may be preferred. Preferably each member is provided with a viewing window or a transparent position or may be completely transparent. Thus in preferred embodiments a user can view the contents of each chamber of the device without having to remove any part of it.

Suitably step (c) involves observing the colour of the tested fluid in each composition/chamber.

The colour intensity could be measured quantitatively using a colour meter. In some preferred embodiments a qualitative assessment of colour is carried out.

In some preferred embodiments a quantative assessment of colour is carried out. This may be carried out by a specially designed apparatus.

According to a third aspect of the present invention there is provided an incubation apparatus comprising multiple recesses each adapted to receive a device of the first aspect.

In some preferred embodiments the incubation apparatus further comprises a spectrophotometer.

Suitably the spectrophotometer is configured to measure the light absorption in each chamber of each device when fitted into the recesses of the incubation apparatus. Suitably the spectrophotometer is configured to measure the absorption of visible light of a particular wavelength in each chamber of each device when fitted into the recesses of the incubation apparatus.

Preferably the incubation apparatus includes a computer which is programmed to measure the light absorption in each chamber of each device repeatedly at specific intervals. Suitably the computer is programmed to record the light absorption in each test chamber of each device and compare this with each control chamber and optionally a standard. This comparison will provide an indication of whether there is any microbial growth in each chamber of each device.

Preferably the incubation apparatus further comprises a display screen. This will display the data in a way to alert the user to in which chamber(s) microbial growth has occurred.

In some embodiments the computer could be configured to indicate whether a particular selective agent is likely to be effective at treating a patient who provided a sample.

According to a fourth aspect of the present invention there is provided a kit comprising an incubation apparatus of the third aspect and one or more devices of the first aspect.

The invention will now be further described with reference to the following non limiting examples and the accompanying drawings in which:

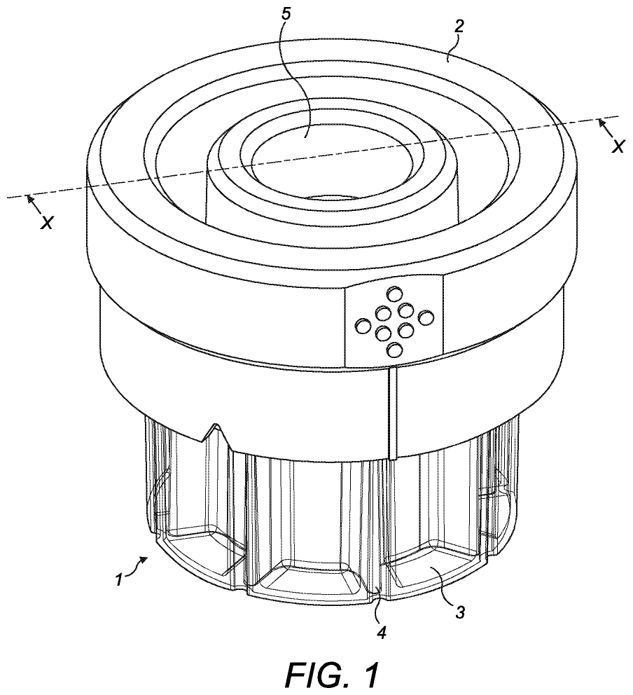

FIG. 1 is a perspective view of a device of the first aspect with the lid in place;

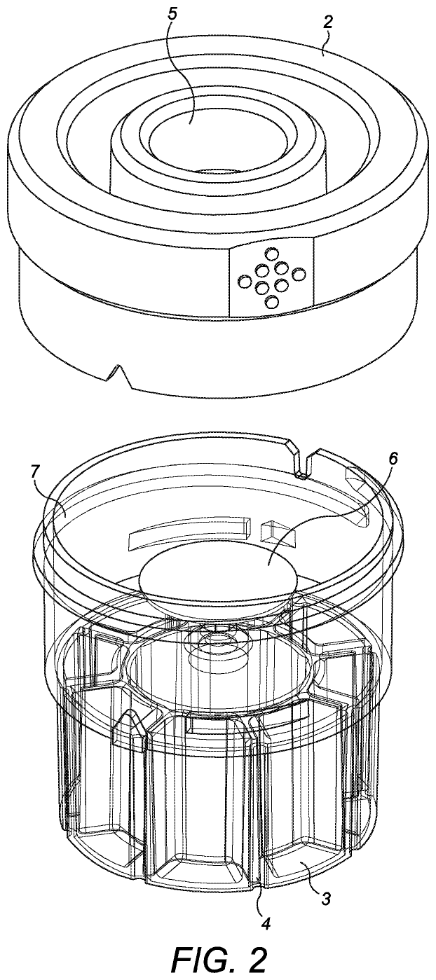

FIG. 2 is an expanded view of the device of FIG. 1 showing the lid removed;

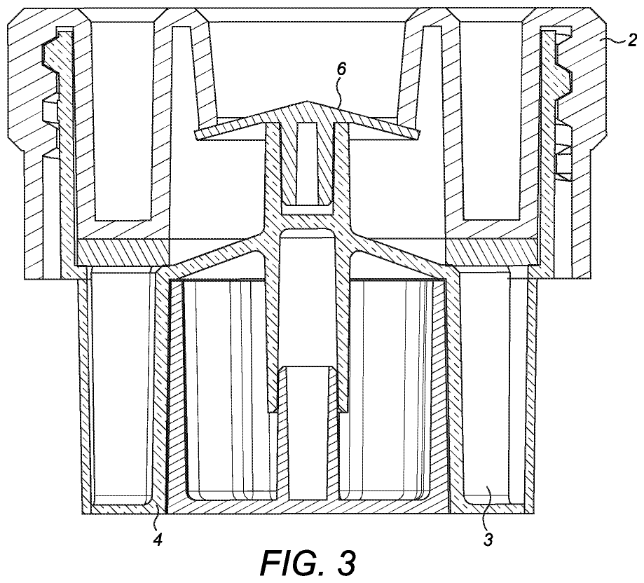

FIG. 3 is a cross sectional view along the plane XX of the device of FIG. 1;

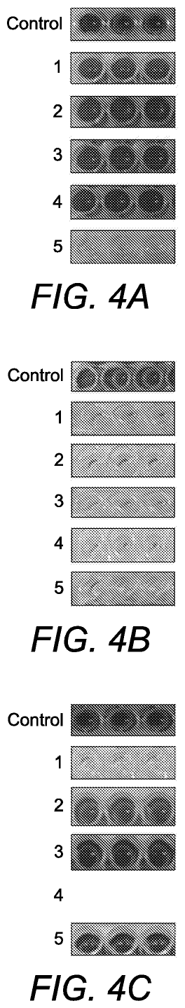

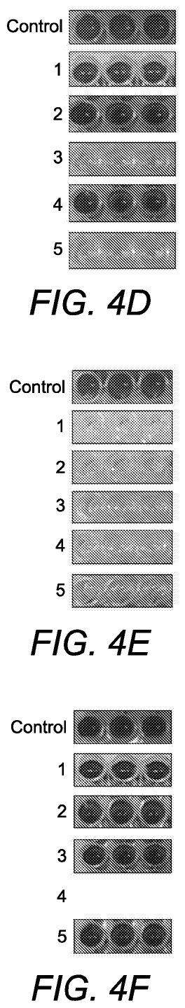

FIGS. 4A to 4F are the results of tested compositions which are later described.

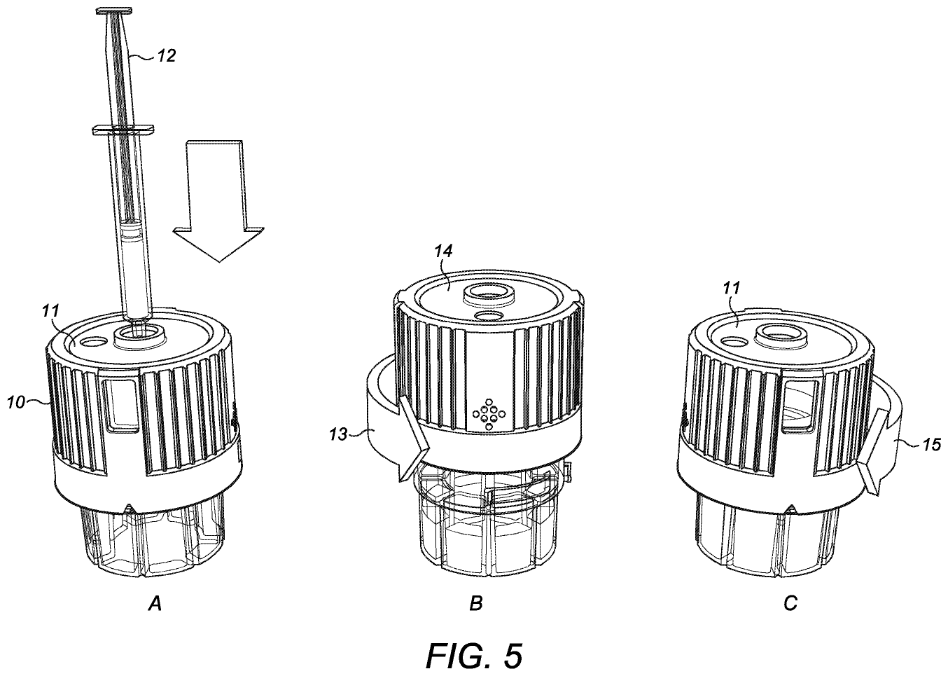

FIG. 5 shows a device of the invention similar to that of FIGS. 1 to 3 which further comprises a dilution reservoir;

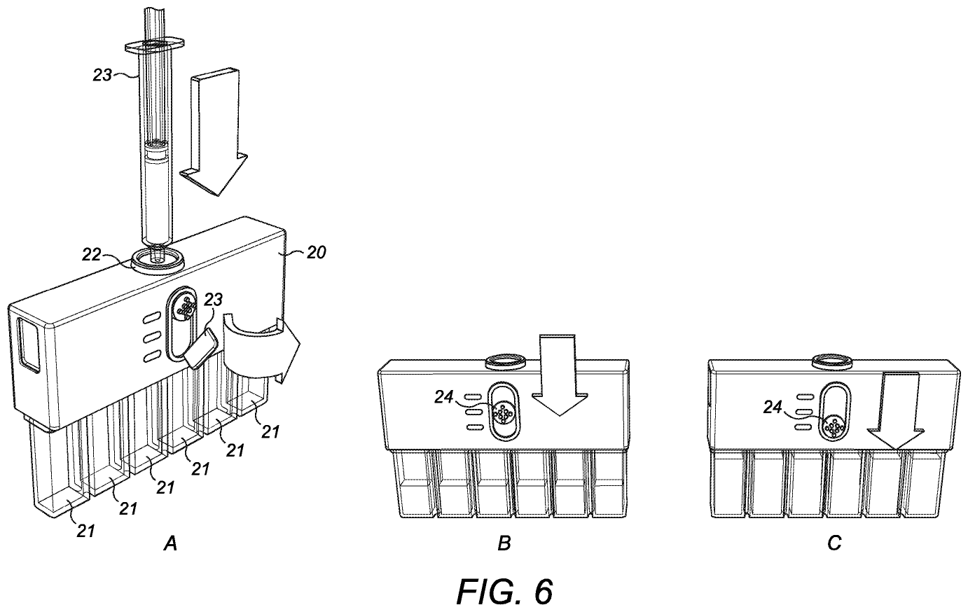

FIG. 6 shows a further device of the invention which comprises a dilution reservoir;

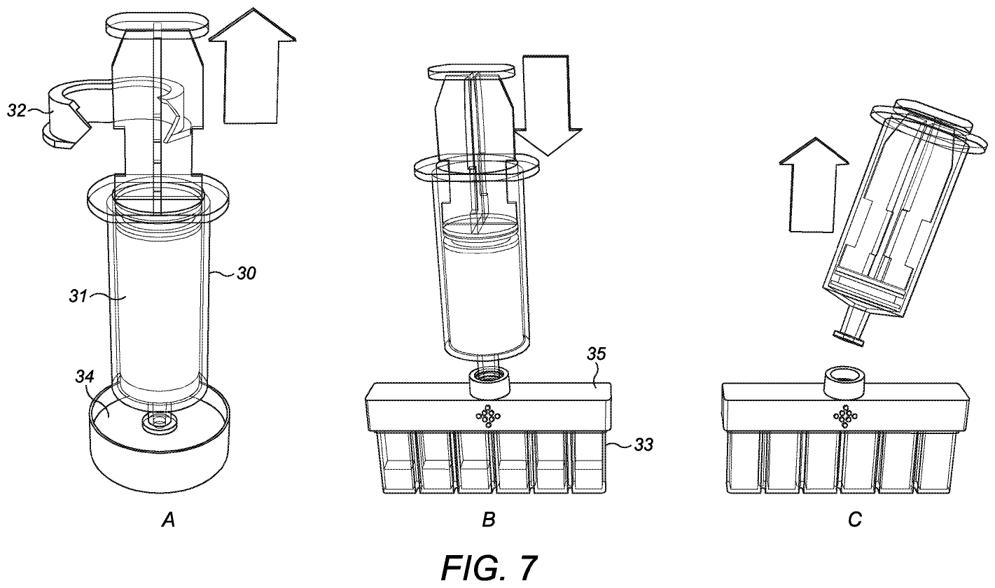

FIG. 7 shows a further device of the invention in which a dilution step is carried out in a syringe, prior to delivering the sample into the device;

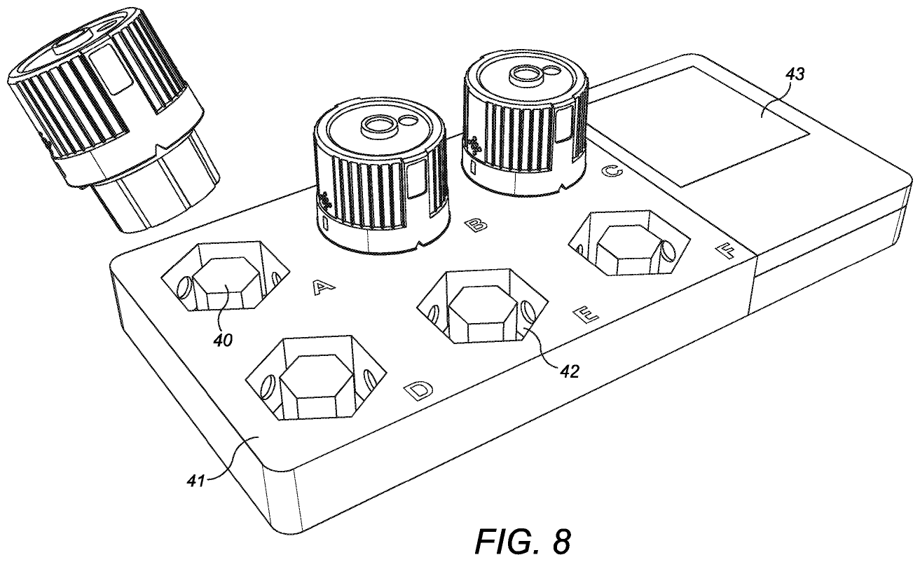

FIG. 8 shows an incubation apparatus comprising recesses adapted to accommodate the device of FIGS. 1 to 3; and

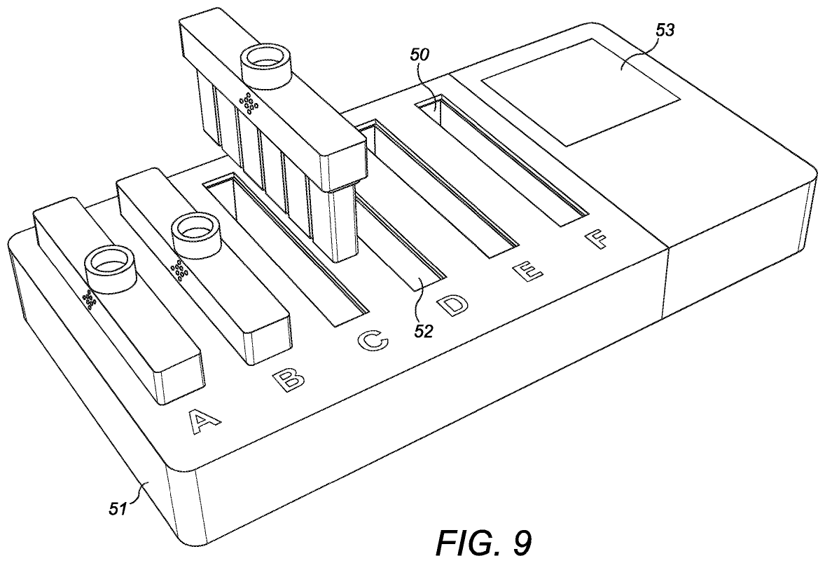

FIG. 9 shows an incubation apparatus comprising recesses adapted to accommodate the device of FIG. 5.

FIGS. 1 to 3 show a device of the present invention including a transparent lower portion 1 for receiving a liquid sample and a lid 2. The lower portion is divided into 8 chambers 3 in an annular arrangement around a central core. An opaque insert 4 (suitably of white plastic) enables the colour of each chamber to be viewed individually without interference from the neighbouring chambers.

Lid 2 includes a central aperture 5. A cap 6 fits into the top of the core of the lower portion. In use when the lid is placed loosely onto the device a liquid poured through aperture 5 is directed by the cap into each of the chambers 3. Once the sample has been poured into the device the lid may be twisted closed by engagement of threads on the internal surface of the lid with threaded region 7 of the transparent lower portion of the device. Closing of the device by the twisting mechanism seals the aperture 5 with the cap 6. This prevents any transfer of liquid between the chambers.

In use each test/control chamber is provided with one or more capsules or other means comprising an indicator compound, nutrients and optionally one or more selective agents.

FIG. 5 shows a device similar to that of FIG. 1 but which further includes a reservoir. The reservoir 10 is fitted to the top of the device in a twistable and lockable manner. A diluent such as a buffer is provided within the reservoir. The reservoir is initially in a locked position (FIG. 5A) as demonstrated by a lock indicator 11. A sample, for example a sample of urine, is delivered into the device via syringe 12.

The reservoir is then twisted as indicated by arrow 13 (FIG. 5B) into a flowing position to allow the now diluted sample to be delivered into each of the chambers. Once the content of the reservoir has been emptied into the chambers the reservoir can be twisted back again (FIG. 5C) in the direction indicated by arrow 15 and the indicator 11 will again show this to be in a locked position. This device can now be incubated.

FIG. 6 shows an alternative device incorporating a reservoir 20 fixed to multiple chambers 21. In use each test/control chamber is provided with one or more capsules or other means comprising an indicator compound, nutrients and optionally one or more selective agents.

A valve 22 is provided at the top of the reservoir and a syringe 23 is used to deliver a sample into the reservoir (FIG. 6A). The reservoir will contain a diluent, for example a buffer solution. A tab 23 maybe used to lock the reservoir in a position such that the contents cannot flow into the chambers while the sample is added. Once the sample has been added to the reservoir the switch 24 is moved down into a flow position (FIG. 6B) to deliver the contents of the reservoir equally into the six chambers shown. When all of the liquid in the reservoir has been delivered into the chambers then the switch 24 is moved into a sealed position (FIG. 6C) in which the contents of the chamber are sealed within them.

FIG. 7 shows a syringe 30 comprising buffer 31 which not locked by a widget 32 to prevent accidental dispensing. A user can withdraw the sample 34 into the syringe (FIG. 7A).

The syringe can then be used to deliver a pre-mixed sample and into a device 33 which includes a portion 35 configured internally to direct flow equally into all of the chambers (FIG. 7B). Once all of the sample has been delivered the syringe can be removed (FIG. 7C).

FIG. 8 shows an incubation apparatus 41 comprising multiple recesses 40 each adapted to receive a device of FIG. 5. Each of the recesses includes multiple optical sensors 42 which measure the absorption at a particular wavelength in each individual chamber. An information screen 43 indicates which particular device unit and which chamber within the device has microbial growth.

FIG. 9 shows an incubation chamber 51 including multiple recesses 50 and an information screen 53. This device is similar to the one disclosed in FIG. 8 but shaped to accommodate the device of FIG. 7.

### EXAMPLES

The device shown in FIGS. 1 and 2 includes 8 chambers. At least one of these is a control chamber which includes an indicator compound and nutrients. One or more of the remaining chambers may be test chambers. One chamber may contain no reagent to show the colour of the urine sample itself.

Each test chamber suitably includes an indicator compound (suitably the same indicator compound as the control chamber, nutrients and one or more selective agents.

The inventors have carried out some experiments in which the same urine sample has been tested (each time in triplicate) in test tubes with compositions suitable for inclusion in the control chamber and test chambers of the invention.

Each test tube contained 0.04 mg tetrazolium violet, 6.6 mg Wilkins Chalgren media. The test chambers also contained an amount of antibiotic compound as indicated in the tables below (as concentration in final volume of urine).

For each example, 0.2 mL of a urine sample was contaminated with 10⁵ cfu/mL of the specified bacteria was poured into each test tube. The resultant composition was incubated at 37° C. for 10 hours. FIGS. 3A to 3F show the test tubes after the incubation period.

Test A - P. aeruginosa

| Chamber | Antibiotic | Concentration (μg/ml) |
|---|---|---|
| Control | None | — |
| Test 1 | Nitrofurantoin | 64 |
| Test 2 | Pivmecillinam | 4 |
| Test 3 | Trimethoprim | 1 |
| Test 4 | Cephalexin | 16 |
| Test 5 | Ciprofloxacin | 0.25 |

The results shown in FIG. 4A indicate that the infection can be treated with ciprofloxacin, and is resistant to the other antibiotics.

Test B - E. coli 1

| Chamber | Antibiotic | Concentration (μg/ml) |
|---|---|---|
| Control | None | — |
| Test 1 | Nitrofurantoin | 64 |
| Test 2 | Pivmecillinam | 4 |
| Test 3 | Trimethoprim | 4 |
| Test 4 | Cephalexin | 16 |
| Test 5 | Ciprofloxacin | 0.06 |

The results shown in FIG. 4B indicate that the infection can be treated with any of the tested antibiotics.

Test C - E. coli 2

| Chamber | Antibiotic | Concentration (μg/ml) |
|---|---|---|
| Control | None | — |
| Test 1 | Nitrofurantoin | 64 |
| Test 2 | Pivmecillinam | 32 |
| Test 3 | Trimethoprim | 32 |
| Test 4 | Cephalexin | — |
| Test 5 | Ciprofloxacin | 0.5 |

The results shown in FIG. 4C indicate that the infection can be treated with nitrofurantoin, and is resistant to the other antibiotics.

Test D - K. pneumoniae

| Chamber | Antibiotic | Concentration (μg/ml) |
|---|---|---|
| Control | None | — |
| Test 1 | Nitrofurantoin | 64 |
| Test 2 | Pivmecillinam | 4 |
| Test 3 | Trimethoprim | 8 |
| Test 4 | Cephalexin | 32 |
| Test 5 | Ciprofloxacin | 0.25 |

The results shown in FIG. 4D indicate that the infection can be treated with ciprofloxacin or trimethoprim, and is resistant to the other antibiotics.

Test E - E. coli 3

| Chamber | Antibiotic | Concentration (μg/ml) |
|---|---|---|
| Control | None | — |
| Test 1 | Nitrofurantoin | 64 |
| Test 2 | Pivmecillinam | 4 |
| Test 3 | Trimethoprim | 1 |
| Test 4 | Cephalexin | 32 |
| Test 5 | Ciprofloxacin | 0.06 |

The results shown in FIG. 4E indicate that the infection can be treated with any of the tested antibiotics.

Test F - P. mirabilis

| Chamber | Antibiotic | Concentration (μg/ml) |
|---|---|---|
| Control | None | — |
| Test 1 | Nitrofurantoin | 64 |
| Test 2 | Pivmecillinam | 16 |
| Test 3 | Trimethoprim | 8 |
| Test 4 | Cephalexin | — |
| Test 5 | Ciprofloxacin | 0.5 |

The results shown in FIG. 4F indicate that the infection cannot be treated with any of the tested antibiotics, and is resistant to all those tested.

1. A device for determining the presence of microorganisms in a sample and the antimicrobial sensitivity thereof, the device comprising multiple chambers including:
a first control chamber; and
a first test chamber;
wherein said first control chamber comprises:
media and/or nutrients that support or encourage microbial growth; and
a tetrazolium indicator compound;
and said first test chamber comprises:
media and/or nutrients that support or encourage microbial growth;
a tetrazolium indicator compound; and
a first selective agent which inhibits the growth and/or replication of one or more specific microorganisms.

2. A method of determining the presence of microorganisms in a sample and the antimicrobial sensitivity thereof, the method comprising:
(a) contacting the sample with a first control composition comprising:
media and/or nutrients that support or encourage microbial growth; and
a tetrazolium indicator compound; and
(b) contacting the sample with a first test composition comprising:
media and/or nutrients that support or encourage microbial growth;
a tetrazolium indicator compound; and
a first selective agent which inhibits the growth and/or replication of one or more specific microorganisms; and
(c) observing said first control composition and said first test composition.

3. A device according to claim 1 wherein the media and/or nutrients that support or encourage microbial growth used in each chamber or composition is selected from brain heart infusion and Wilkins Chalgren media.

4. A device according to claim 1 wherein the tetrazolium compound used in each chamber or composition is tetrazolium violet.

5. A device according to claim 1 wherein the first selective agent is selected from ciprofloxacin, nitrofurantoin, pivmecillnam, trimethoprim and cephalexin.

6. A device of claim 1 which further comprises one or more further test chambers wherein the or each further test chamber comprises media and/or nutrients that support or encourage microbial growth; a tetrazolium indicator compound; and a selective agent which inhibits the growth and/or replication of one or more specific microorganisms.

7. A method according to claim 2 which further comprises contacting the sample with one or more further test compositions wherein the or each further test composition comprises: media and/or nutrients that support or encourage microbial growth; a tetrazolium indicator compound; and a selective agent which inhibits the growth and/or replication of one or more specific microorganisms.

8. A device according to claim 6 wherein each chamber or each composition comprises the same media and/or nutrients that support and/or encourage microbial growth and the same indicator compound.

9. A device according to claim 6, wherein each test chamber or each test composition comprises a different selective agent or combination of selective agents which inhibits the growth and/or replication of one or more specific microorganisms.

10. A device according to claim 9 wherein each selective agent is selected from ciprofloxacin, nitrofurantoin, pivmecillinam, trimethoprim and cephalexin.

11. A method according to claim 2 wherein the sample is a urine sample.

12. A method according to claim 7 wherein step (c) involves observing the colour of the control composition(s) and test composition(s).

13. A method according to claim 2 wherein the compositions are incubated after steps (a) and (b) and before step (c).

14. A device according to claim 1 which further comprises a dilution reservoir.

15. A device for determining the presence of microorganisms in a sample and the antimicrobial sensitivity thereof, preferably a urine sample, the device comprising: a first control chamber and five test chambers wherein each chamber comprises media and/or nutrients that support or encourage microbial growth and a tetrazolium indicator compound and each test chamber comprises a selective agent; wherein the first test chamber comprises ciprofloxacin as the selective agent, the second test chamber comprises nitrofurantoin as the selective agent, the third test chamber comprises pivmecillinam as the selective agent, the fourth test chamber comprises trimethoprim as the selective agent and the fifth test chamber comprises cephalexin as the selective agent.

16. A method according to claim 2 which includes a step prior to step (a) and step (b) of diluting the sample.

17. (canceled)

18. An incubation apparatus comprising multiple recesses each adapted to receive a device of claim 1.

19. A kit comprising an incubation apparatus according to claim.
